(19) United States
(12) Patent Application Publication
(10) Pub. No.: US 2014/0371243 A1
(43) Pub. Date: Dec. 18, 2014

(54) MEDICAL USE OF A DPP-4 INHIBITOR

(71) Applicants: Thomas KLEIN, Radolfzell (DE); Takashi NOMIYAMA, Jonan-ku (JP); Yuichi TERAWAKI, Jonan-ku (JP); Toshihiko YANASE, Jonan-ku (JP)

(72) Inventors: Thomas KLEIN, Radolfzell (DE); Takashi NOMIYAMA, Jonan-ku (JP); Yuichi TERAWAKI, Jonan-ku (JP); Toshihiko YANASE, Jonan-ku (JP)

(73) Assignee: Boehringer Ingelheim International GmbH, Ingelheim am Rhein (DE)

(21) Appl. No.: 14/303,852

(22) Filed: Jun. 13, 2014

(30) Foreign Application Priority Data
Jun. 14, 2013 (EP) 13003042.2

(51) Int. Cl. A61K 31/522; A61K 31/517; A61K 45/06

(52) U.S. Cl. CPC A61K 31/522; A61K 45/06; A61K 31/517
USPC 514/263.21; 514/266.22

(57) ABSTRACT

The present invention relates to the use of a certain DPP-4 inhibitor along with angioplasty or stenting, and/or to its use for treating and/or preventing restenosis from angioplasty or stenting.

### FIELD OF THE INVENTION

The present invention relates to a certain DPP-4 inhibitor (preferably linagliptin), optionally in combination with one or more other active agents, for use in patients who are in need of, indicated for or have undergone angioplasty and/or stenting.

The present invention further relates to a certain DPP-4 inhibitor (preferably linagliptin), optionally in combination with one or more other therapeutic agents and/or principles, such as e.g. angioplasty or stents (e.g. peripheral or coronary stents) including bare-metal stents or drug-eluting stents (e.g. such stents releasing a drug to block cell proliferation), for use in treating and/or preventing stenosis, (large) blood vessel narrowing or re-narrowing, revascularization or restenosis (and/or treating, preventing, reducing the risk of, slowing progression of, delaying the onset/occurrence/re-occurrence of, and/or protecting against stenosis, blood vessel narrowing, re-narrowing or occlusion, revascularization or restenosis, blood clots, neointimal hyperplasia and/or major adverse cardiac events (MACE, such as e.g. death, myocardial infarction or repeat intervention because of restenosis), e.g. following or associated with (peripheral or coronary) angioplasty (such as e.g. performed with stents, stent implantations, bypass operations or balloon catheters), to pharmaceutical compositions or combinations comprising such therapeutic components, and to certain therapeutic uses thereof.

### BACKGROUND OF THE INVENTION

Atherosclerosis, the subsequent development of occlusive vascular diseases, and the failure of treatment approaches such as post-angioplasty re-stenosis involve several interrelated processes.

In addition to endothelial dysfunction and inflammation, proliferation of smooth muscle cells (SMC) is considered to play a pivotal role in the pathogenesis of atherosclerosis and the failure or complications of interventional approaches used to treat related occlusive vascular complications, especially in diabetes mellitus.

A problem that can occur after angioplasty is too much tissue growth (such as e.g. neointima formation, SMC proliferation, neointimal hyperplasia) within the treated portion of the vessel (such as tissue growth in or around the stent used in angioplasty). This can cause the vessel to become narrow or blocked again, often within 6 months. This complication is known as restenosis.

Incretin based therapy has emerged as a promising treatment for type 2 diabetes. Recently, attention has been focused on incretins because of their tissue-protective effects in addition to their glucose lowering role.

Patients with diabetes mellitus have a higher risk of cardiovascular events compared with non-diabetic patients and also frequently fail re-stenosis following coronary angioplasty, even when the intervention is performed with drug-eluting stents.

It has been investigated whether an incretin-related antidiabetes drug could improve cardiovascular systems.

The vascular-protective effects of exendin-4, a glucagon-like peptide (GLP)-1 receptor agonist, have been demonstrated.

It has been previously reported the reduction of intimal thickening after vascular injury via 5′ adenosine monophosphate-activated protein kinase activation in vascular SMC1.

However, there are no data investigating whether dipeptidyl peptidase (DPP)-4 inhibition may directly attenuate neointima formation and SMC proliferation.

### SUMMARY OF THE INVENTION

The present invention relates to a certain DPP-4 inhibitor (preferably linagliptin), optionally in combination with one or more other active agents, for use along with angioplasty and/or stenting.

The present invention relates to a certain DPP-4 inhibitor (preferably linagliptin), optionally in combination with one or more other active agents, for use in preventing restenosis in angioplasty or stenting.

The present invention further relates to the medical use of a certain DPP-4 inhibitor along with angioplasty or stenting, and/or to its use for treating and/or preventing restenosis from angioplasty or stenting.

The present invention relates to a certain DPP-4 inhibitor (preferably linagliptin) for use in treating, preventing and/or reducing the risk of stenosis, (large) blood vessel narrowing or re-narrowing, revascularization or restenosis (and/or treating, preventing, reducing the risk of, slowing progression of, delaying the onset/occurrence/re-occurrence of, and/or protecting against stenosis, blood vessel narrowing, re-narrowing or occlusion, revascularization or restenosis, blood clots, neointimal hyperplasia and/or major adverse cardiac events (MACE, such as e.g. death, myocardial infarction or repeat intervention because of restenosis), e.g. following or associated with (peripheral or coronary) angioplasty (such as e.g. performed with stents, stent implantations, bypass operations or balloon cathetering), optionally in combination with one or more other active agents.

The present invention contemplates a certain DPP-4 inhibitor (preferably linagliptin), optionally in combination with one or more other active agents, for treating, preventing and/or reducing the risk of complications (e.g. restenosis, blood clots, neointimal hyperplasia and/or MACE) from angioplasty and/or stenting.

The present invention further contemplates a certain DPP-4 inhibitor (preferably linagliptin), optionally in combination with one or more other active agents, for treating, preventing and/or reducing the risk of in-stent restenosis (ISR) and/or post-angioplasty restenosis (PARS).

Further, the invention relates to a DPP-4 inhibitor, particularly linagliptin, optionally in combination with a stent, e.g. for use in preparing a pharmaceutical composition, kit, medical product or device (e.g. a drug-eluting stent, such as e.g. a stent comprising a drug-release film coat containing the DPP-4 inhibitor), which can be used to treat, prevent and/or reduce the risk of complications (e.g. restenosis, blood clots, neointimal hyperplasia and/or MACE) from stenting.

Further, the invention relates to a drug-eluting stent (e.g. for implantation or placement at a site of vascular injury) containing a DPP-4 inhibitor, particularly linagliptin, and optionally one or more pharmaceutically acceptable carriers (such as e.g. a stent comprising a drug-release film coat containing the DPP-4 inhibitor for release), such as e.g. for treating, preventing and/or reducing the risk of complications (e.g. restenosis, blood clots, neointimal hyperplasia and/or MACE) from stenting.

Further, the invention relates to a method of treating, preventing and/or reducing the risk of complications (e.g. restenosis, blood clots, neointimal hyperplasia and/or MACE) associated with or following angioplasty and/or stenting, said method comprising administering or applying an effective amount of a DPP-4 inhibitor, particularly linagliptin, optionally in combination with one or more other therapeutic agents and/or principles, such as e.g. a stent, to the patient in need thereof.

Further, the invention relates to a method of treating, preventing and/or reducing the risk of complications (e.g. restenosis, blood clots, neointimal hyperplasia and/or MACE), which may be associated with or following stenting, said method comprising administering an effective amount of a DPP-4 inhibitor, particularly linagliptin, and optionally one or more other active agents, to the patient in need thereof (who may be a stented patient).

Further, the invention relates to a method of treating, preventing and/or reducing the risk of complications (e.g. restenosis, blood clots, neointimal hyperplasia and/or MACE) associated with or following stenting, said method comprising applying the stent and administering an effective amount of a DPP-4 inhibitor, particularly linagliptin, and optionally one or more other therapeutic agents, to the patient in need thereof.

Further, the invention relates to a method of treating, preventing and/or reducing the risk of complications (e.g. restenosis, blood clots, neointimal hyperplasia and/or MACE) associated with or following stenting, said method comprising administering or applying a stent containing an effective amount of a DPP-4 inhibitor, particularly linagliptin, and optionally one or more other therapeutic agents, to the patient in need thereof.

Further, the invention relates to a combination of a DPP-4 inhibitor, particularly linagliptin, and a stent, and optionally one or more other therapeutic agents, such as e.g. for use in treating, preventing and/or reducing the risk of complications (e.g. restenosis, blood clots, neointimal hyperplasia and/or MACE) associated with or following stenting.

Other aspects of the present invention become apparent to the skilled person from the foregoing and following remarks (including the examples and claims).

### BRIEF DESCRIPTION OF THE DRAWINGS

FIGS. 1A and 1B: Linagliptin vs. Control does not change body weight or blood glucose in non-diabetic mice.

FIG. 2: Linagliptin vs. Control increases serum active GLP-1 concentration (ELISA) in non-diabetic mice.

FIGS. 3A, 3B, and 3C: Linagliptin vs. Control attenuates neointima formation in femoral arteries (tissue analysis) after vascular injury in non-diabetic mice (guide wire and elastic staining, intima area, media area, and intima/media ratio)

FIG. 4: Linagliptin reduces FBS-induced vascular smooth muscle (SMC) proliferation in vitro (BrdU assay).

### DETAILED DESCRIPTION OF THE INVENTION

Within the scope of the present invention it has been found that certain DPP-4 inhibitors, preferably linagliptin, optionally in combination with one or more other active agents or therapeutic principles (such as e.g. a stent), each as described herein, have properties which make them suitable for the purpose of this invention.

For example, the dipeptidyl peptidase (DPP)-4 inhibitor linagliptin has been found to attenuate neointima formation and SMC proliferation, and is therefore useful for treating and/or preventing neointimal hyperplasia and restenosis.

DPP-4 is analogous to CD26 a T-cell antigene which plays a role in T-cell activation and immuno-modulation. Furthermore, linagliptin, a selective DPP-4 inhibitor further qualifies for the instant purposes with certain anti-oxidative and/or anti-inflammatory features.

In an embodiment, the patient described herein is a diabetic patient (particularly human), such as having diabetes (e.g. type 1 or type 2 diabetes or LADA, particularly type 2 diabetes).

In another embodiment, the patient described herein is a non-diabetic patient (particularly human), such as without diabetes (e.g. type 1 or type 2 diabetes or LADA, particularly type 2 diabetes).

In a further embodiment, the patient described herein is (diabetic or non-diabetic) patient (particularly human patient) who is in need of or who is indicated for or who has (previously) undergone angioplasty (e.g. peripheral or coronary angioplasty), such as e.g. performed with stents, stent implantations, bypass operations or balloon catheters.

Thus, the present invention provides a certain DPP-4 inhibitor (particularly linagliptin) for use along with angioplasty or stenting.

The present invention further provides a certain DPP-4 inhibitor (particularly linagliptin) for use in preventing restenosis in angioplasty or stenting.

The present invention further relates to a certain DPP-4 inhibitor (particularly linagliptin), optionally in combination with one or more other active agents, for treating:

a patient (particularly human patient) who is in need of, who is indicated for or who has (previously) undergone (peripheral or coronary) angioplasty (such as e.g. performed with stents, stent implantations, bypass operations or balloon catheters); and/or
a patient (particularly human patient) having or being at risk of restenosis, e.g. following or associated with (peripheral or coronary) angioplasty (such as e.g. performed with stents, stent implantations, bypass operations or balloon catheters); and/or
a patient (particularly human patient) having or being at risk of stenosis, blood vessel narrowing, re-narrowing or occlusion, revascularization or restenosis, blood clots, neointimal hyperplasia and/or major adverse cardiac events (MACE), such as e.g. death, myocardial infarction or repeat intervention because of restenosis), e.g. following or associated with (peripheral or coronary) angioplasty (such as e.g. performed with stents, stent implantations, bypass operations or balloon catheters).

The present invention further relates to a pharmaceutical composition or combination comprising or consisting essentially of a certain DPP-4 inhibitor (particularly linagliptin), optionally in combination or alternation with one or more other therapeutic agents or principles (such as e.g. angioplasty or a stent), each as described herein, such as e.g. for simultaneous, sequential or separate medical use in therapy or prophylaxis.

The present invention further relates to a medical combination comprising or consisting essentially of (coronary or peripheral) angioplasty (such as e.g. to be performed with stents, stent implantations, bypass operations or balloon catheters) and a certain DPP-4 inhibitor (particularly linagliptin), and optionally one or more other active agents, each as described herein, such as e.g. for simultaneous, sequential or separate medical use in therapy or prophylaxis.

The present invention further relates to a method for treating, preventing, reducing the risk of, slowing progression of, delaying the onset/occurrence/re-occurrence of, and/or protecting against stenosis, blood vessel narrowing, re-narrowing or occlusion, revascularization or restenosis, blood clots, neointimal hyperplasia and/or major adverse cardiac events (MACE, such as e.g. death, myocardial infarction or repeat intervention because of restenosis) in a patient (particularly human patient) in need thereof, said method comprising administering or applying an effective amount of a certain DPP-4 inhibitor (particularly linagliptin), optionally in combination with one or more other therapeutic agents or principles (such as e.g. angioplasty, such as stenting), each as described herein, to the patient.

The present invention further relates to a method for treating a patient with indication for angioplasty (such as e.g. to be performed with stents, stent implantations, bypass operations or balloon catheters), said method comprising applying angioplasty in combination with administering or applying an effective amount of a certain DPP-4 inhibitor (particularly linagliptin), and optionally one or more other therapeutic agents, to the patient.

The present invention further relates to a method of treating, preventing and/or reducing the risk of complications (e.g. restenosis, blood clots, neointimal hyperplasia and/or MACE) associated with or following angioplasty (such as e.g. performed with stents, stent implantations, bypass operations or balloon catheters), said method comprising administering or applying an effective amount of a certain DPP-4 inhibitor (particularly linagliptin), optionally in combination with one or more other therapeutic agents or principles, to the patient.

The present invention further relates to a method of applying angioplasty (such as e.g. performed with stents, stent implantations, bypass operations or balloon catheters) to a patient, said method comprising applying angioplasty, and administering or applying an effective amount of a certain DPP-4 inhibitor (particularly linagliptin) and optionally one or more other therapeutic agents, to the patient.

Within this invention it is to be understood that the combinations or combined uses according to this invention may envisage the simultaneous, sequential or separate administration of the therapeutic components.

In this context, “combination” or “combined” within the meaning of this invention may include, without being limited, fixed and non-fixed (e.g. free) forms (including kits, or other administration, application or dosage forms) and uses, such as e.g. the simultaneous, sequential or separate use of the components.

The combined administration or application of this invention may take place by administering or applying the therapeutic components together, such as e.g. by administering or applying them simultaneously in one single or in two separate formulations or forms. Alternatively, the administration or application may take place by administering or applying the therapeutic components sequentially, such as e.g. successively in two separate formulations or forms.

For the combination therapy of this invention the therapeutic components may be administered or applied separately (which implies that they are formulated separately) or formulated altogether (which implies that they are formulated in the same preparation or in the same form). Hence, the administration of one element of the combination of the present invention may be prior to, concurrent to, or subsequent to the administration of the other element of the combination.

A DPP-4 inhibitor within the meaning of the present invention includes, without being limited to, any of those DPP-4 inhibitors mentioned hereinabove and hereinbelow, preferably orally active DPP-4 inhibitors. In a further embodiment, a DPP-4 inhibitor within the meaning of the present invention includes preferably orally and/or subcutaneously and/or topically active DPP-4 inhibitors.

In a first embodiment (embodiment A), a DPP-4 inhibitor in the context of the present invention is any DPP-4 inhibitor of

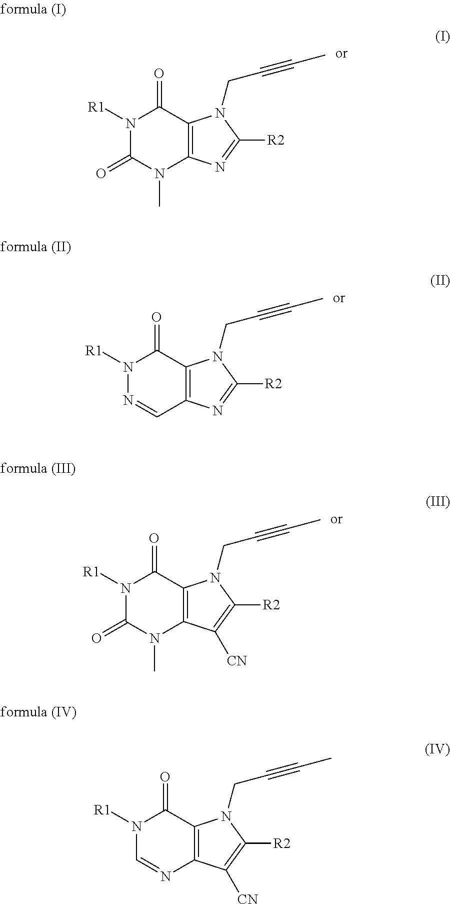

wherein R1 denotes ([1,5]naphthyridin-2-yl)methyl, (quinazolin-2-yl)methyl, (quinoxalin-6-yl)methyl, (4-methyl-quinazolin-2-yl)methyl, 2-cyano-benzyl, (3-cyano-quinolin-2-yl)methyl, (3-cyano-pyridin-2-yl)methyl, (4-methyl-pyrimidin-2-yl)methyl, or (4,6-dimethyl-pyrimidin-2-yl)methyl and R2 denotes 3-(R)-amino-piperidin-1-yl, (2-amino-2-methyl-propyl)-methylamino or (2-(S)-amino-propyl)-methylamino,
or its pharmaceutically acceptable salt.

Regarding the first embodiment (embodiment A), preferred DPP-4 inhibitors are any or all of the following compounds and their pharmaceutically acceptable salts:
1-[(4-methyl-quinazolin-2-yl)methyl]-3-methyl-7-(2-butyn-1-yl)-8-(3-(R)-amino-piperidin-1-yl)-xanthine (compare WO 2004/018468, example 2(142)):

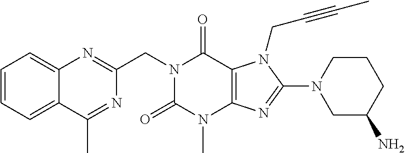 [SMILES: CC#CCn1c(N2CCC[C@@H](N)C2)nc2c1c(=O)n(Cc1nc(C)c3ccccc3n1)c(=O)n2C]

1-[([1,5]naphthyridin-2-yl)methyl]-3-methyl-7-(2-butyn-1-yl)-8-((R)-3-amino-piperidin-1-yl)-xanthine (compare WO 2004/018468, example 2(252)):

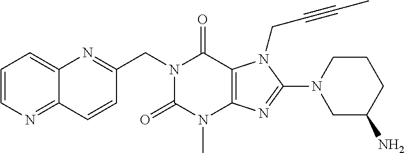 [SMILES: CC#CCn1c(N2CCC[C@@H](N)C2)nc2c1c(=O)n(Cc1ccc3ncccc3n1)c(=O)n2C]

1-[(Quinazolin-2-yl)methyl]-3-methyl-7-(2-butyn-1-yl)-8-((R)-3-amino-piperidin-1-yl)-xanthine (compare WO 2004/018468, example 2(80)):

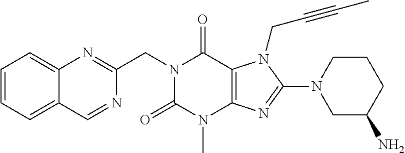 [SMILES: CC#CCn1c(N2CCC[C@@H](N)C2)nc2c1c(=O)n(Cc1ncc3ccccc3n1)c(=O)n2C]

2-((R)-3-Amino-piperidin-1-yl)-3-(but-2-yinyl)-5-(4-methyl-quinazolin-2-ylmethyl)-3,5-dihydro-imidazo[4,5-d]pyridazin-4-one (compare WO 2004/050658, example 136):

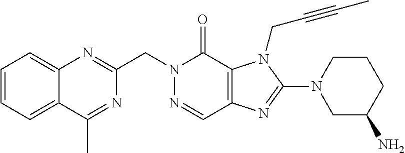 [SMILES: CC#CCn1c(N2CCC[C@@H](N)C2)nc2cnn(Cc3nc(C)c4ccccc4n3)c(=O)c21]

1-[(4-Methyl-quinazolin-2-yl)methyl]-3-methyl-7-(2-butyin-1-yl)-8-[(2-amino-2-methyl-propyl)-methylamino]-xanthine (compare WO 2006/029769, example 2(1)):

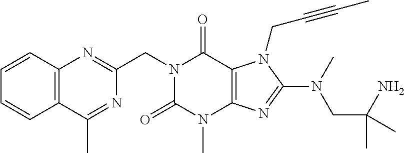 [SMILES: CC#CCn1c(N(C)CC(C)(C)N)nc2c1c(=O)n(Cc1nc(C)c3ccccc3n1)c(=O)n2C]

1-[(3-Cyano-quinolin-2-yl)methyl]-3-methyl-7-(2-butyn-1-yl)-8-((R)-3-amino-piperidin-1-yl)-xanthine (compare WO 2005/085246, example 1(30)):

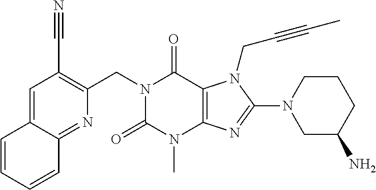 [SMILES: CC#CCn1c(N2CCC[C@@H](N)C2)nc2c1c(=O)n(Cc1nc3ccccc3cc1C#N)c(=O)n2C]

1-(2-Cyano-benzyl)-3-methyl-7-(2-butyn-1-yl)-8-((R)-3-amino-piperidin-1-yl)-xanthine (compare WO 2005/085246, example 1(39)):

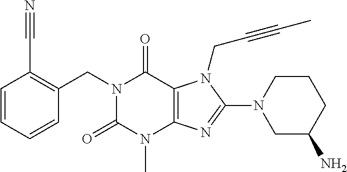 [SMILES: CC#CCn1c(N2CCC[C@@H](N)C2)nc2c1c(=O)n(Cc1ccccc1C#N)c(=O)n2C]

1-[(4-Methyl-quinazolin-2-yl)methyl]-3-methyl-7-(2-butyn-1-yl)-8-[(S)-(2-amino-propyl)-methylamino]-xanthine (compare WO 2006/029769, example 2(4)):

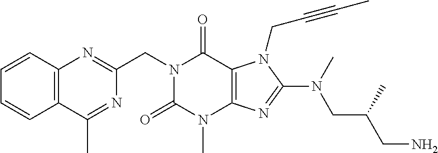 [SMILES: CC#CCn1c(N(C)C[C@H](C)CN)nc2c1c(=O)n(Cc1nc(C)c3ccccc3n1)c(=O)n2C]

1-[(3-Cyano-pyridin-2-yl)methyl]-3-methyl-7-(2-butyn-1-yl)-8-((R)-3-amino-piperidin-1-yl)-xanthine (compare WO 2005/085246, example 1(52)):

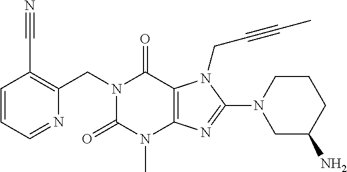 [SMILES: CC#CCn1c(N2CCC[C@@H](N)C2)nc2c1c(=O)n(Cc1ncccc1C#N)c(=O)n2C]

1-[(4-Methyl-pyrimidin-2-yl)methyl]-3-methyl-7-(2-butyn-1-yl)-8-((R)-3-amino-piperidin-1-yl)-xanthine (compare WO 2005/085246, example 1(81)):

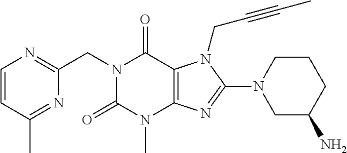 [SMILES: CC#CCn1c(N2CCC[C@@H](N)C2)nc2c1c(=O)n(Cc1nccc(C)n1)c(=O)n2C]

1-[(4,6-Dimethyl-pyrimidin-2-yl)methyl]-3-methyl-7-(2-butyn-1-yl)-8-((R)-3-amino-piperidin-1-yl)-xanthine (compare WO 2005/085246, example 1(82)):

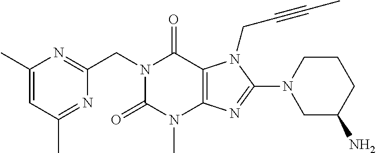 [SMILES: CC#CCn1c(N2CCC[C@@H](N)C2)nc2c1c(=O)n(Cc1nc(C)cc(C)n1)c(=O)n2C]

1-[(Quinoxalin-6-yl)methyl]-3-methyl-7-(2-butyn-1-yl)-8-((R)-3-amino-piperidin-1-yl)-xanthine (compare WO 2005/085246, example 1(83)):

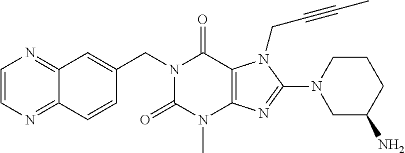 [SMILES: CC#CCn1c(N2CCC[C@@H](N)C2)nc2c1c(=O)n(Cc1ccc3nccnc3c1)c(=O)n2C]

These DPP-4 inhibitors are distinguished from structurally comparable DPP-4 inhibitors, as they combine exceptional potency and a long-lasting effect with favourable pharmacological properties, receptor selectivity and a favourable side-effect profile or bring about unexpected therapeutic advantages or improvements when combined with other pharmaceutical active substances. Their preparation is disclosed in the publications mentioned.

In a second embodiment (embodiment B), a DPP-4 inhibitor in the context of the present invention is a DPP-4 inhibitor selected from the group consisting of

sitagliptin, vildagliptin, saxagliptin, alogliptin, gemigliptin, omarigliptin, evogliptin,
(2S)-1-{[2-(5-Methyl-2-phenyl-oxazol-4-yl)-ethylamino]-acetyl}-pyrrolidine-2-carbonitrile,
(2S)-1-{[1,1,-Dimethyl-3-(4-pyridin-3-yl-imidazol-1-yl)-propylamino]-acetyl}-pyrrolidine-2-carbonitrile,
(S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one,
(3,3-Difluoropyrrolidin-1-yl)-((2S,4S)-4-(4-(pyrimidin-2-yl)piperazin-1-yl)pyrrolidin-2-yl)methanone,
(1((3S,4S)-4-amino-1-(4-(3,3-difluoropyrrolidin-1-yl)-1,3,5-triazin-2-yl)pyrrolidin-3-yl)-5,5-difluoropiperidin-2-one,
(2S,4S)-1-{2-[(3S,1R)-3-(1H-1,2,4-Triazol-1-ylmethyl)cyclopentylamino]-acetyl}-4-fluoropyrrolidine-2-carbonitrile,
(R)-2-[6-(3-Amino-piperidin-1-yl)-3-methyl-2,4-dioxo-3,4-dihydro-2H-pyrimidin-1-ylmethyl]-4-fluoro-benzonitrile,
5-{(S)-2-[2-((S)-2-Cyano-pyrrolidin-1-yl)-2-oxo-ethylamino]-propyl}-5-(1H-tetrazol-5-yl)-10,11-dihydro-5H-dibenzo[a,d]cycloheptene-2,8-dicarboxylic acid bis-dimethylamide,
3-{(2S,4S)-4-[4-(3-Methyl-1-phenyl-1H-pyrazol-5-yl)piperazin-1-yl]pyrrolidin-2-ylcarbonyl}thiazolidine,
[(2R)-1-{[(3R)-pyrrolidin-3-ylamino]acetyl}pyrrolidin-2-yl]boronic acid,
(2S,4S)-1-[2-[(4-ethoxycarbonylbicyclo[2.2.2]oct-1-yl)amino]acetyl]-4-fluoropyrrolidine-2-carbonitrile,
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile,
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione, and
(S)-2-methylpyrazolo[1,5-a]primidine-6-carboxylic acid {2-[(2-cyanopyrrolidin-1-yl)-2-oxoethylamino]-2-methylpropyl}amide,
   or its pharmaceutically acceptable salt.

A more preferred DPP-4 inhibitor among the abovementioned DPP-4 inhibitors of embodiment A of this invention is 1-[(4-methyl-quinazolin-2-yl)methyl]-3-methyl-7-(2-butyn-1-yl)-8-(3-(R)-amino-piperidin-1-yl)-xanthine, particularly the free base thereof (which is also known as linagliptin or BI 1356).

Preferably the DPP-4 inhibitor of this invention is selected from the group consisting of linagliptin, sitagliptin, vildagliptin, alogliptin, saxagliptin, teneligliptin, anagliptin, gemigliptin and dutogliptin, or a pharmaceutically acceptable salt of one of the herein mentioned DPP-4 inhibitors, or a prodrug thereof.

A particularly preferred DPP-4 inhibitor to be emphasized within the present invention is linagliptin. The term “linagliptin” as employed herein refers to linagliptin or a pharmaceutically acceptable salt thereof, including hydrates and solvates thereof, and crystalline forms thereof, preferably linagliptin refers to 1-[(4-methyl-quinazolin-2-yl)methyl]-3-methyl-7-(2-butyn-1-yl)-8-(3-(R)-amino-piperidin-1-yl)-xanthine. Crystalline forms are described in WO 2007/128721. Methods for the manufacture of linagliptin are described in the patent applications WO 2004/018468 and WO 2006/048427 for example. Linagliptin is distinguished from structurally comparable DPP-4 inhibitors, as it combines exceptional potency and a long-lasting effect with favourable pharmacological properties, receptor selectivity and a favourable side-effect profile or bring about unexpected therapeutic advantages or improvements in therapy.

With respect to embodiment A, the methods of synthesis for the DPP-4 inhibitors according to embodiment A of this invention are known to the skilled person. Advantageously, the DPP-4 inhibitors according to embodiment A of this invention can be prepared using synthetic methods as described in the literature. Thus, for example, purine derivatives of formula (I) can be obtained as described in WO 2002/068420, WO 2004/018468, WO 2005/085246, WO 2006/029769 or WO 2006/048427, the disclosures of which are incorporated herein. Purine derivatives of formula (II) can be obtained as described, for example, in WO 2004/050658 or WO 2005/110999, the disclosures of which are incorporated herein. Purine derivatives of formula (III) and (IV) can be obtained as described, for example, in WO 2006/068163, WO 2007/071738 or WO 2008/017670, the disclosures of which are incorporated herein. The preparation of those DPP-4 inhibitors, which are specifically mentioned hereinabove, is disclosed in the publications mentioned in connection therewith. Polymorphous crystal modifications and formulations of particular DPP-4 inhibitors are disclosed in WO 2007/128721 and WO 2007/128724, respectively, the disclosures of which are incorporated herein in their entireties. Formulations of particular DPP-4 inhibitors with metformin or other combination partners are described in WO 2009/121945, the disclosure of which is incorporated herein in its entirety.

With respect to embodiment B, the methods of synthesis for the DPP-4 inhibitors of embodiment B are described in the scientific literature and/or in published patent documents, particularly in those cited herein.

In an embodiment, the DPP-4 inhibitor according to the invention is preferably administered orally.

In further embodiment, the DPP-4 inhibitor according to the invention is used in combination with angioplasty.

In further embodiment, the DPP-4 inhibitor according to the invention is used with angioplasty such as stent implantation, bypass operation or balloon cathetering procedure, wherein the DPP-4 inhibitor is preferably administered orally.

In further embodiment, the DPP-4 inhibitor according to the invention is administered (preferably orally) to a patient in combination with angioplasty or stenting procedure.

In another embodiment, the DPP-4 inhibitor according to the invention is applied together with a stent, e.g. as drug-coated or drug-releasing stent containing the DPP-4 inhibitor, for example as an implantable stent coated with the DPP-4 inhibitor such as for local release in the vessel.

Suitable doses and dosage forms of the DPP-4 inhibitors may be determined by a person skilled in the art and may include those described herein or in the relevant references.

For pharmaceutical application in warm-blooded vertebrates, particularly humans, the compounds of this invention are usually used in dosages from 0.001 to 100 mg/kg body weight, preferably at 0.01-15 mg/kg or 0.1-15 mg/kg, in each case 1 to 4 times a day. For this purpose, the compounds, optionally combined with other active substances, may be incorporated together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof into conventional galenic preparations such as plain or coated tablets, capsules, powders, suspensions or suppositories.

The pharmaceutical compositions according to this invention comprising the DPP-4 inhibitors as defined herein are thus prepared by the skilled person using pharmaceutically acceptable formulation excipients as described in the art and appropriate for the desired route of administration. Examples of such excipients include, without being restricted to diluents, binders, carriers, fillers, lubricants, flow promoters, crystallisation retardants, disintegrants, solubilizers, colorants, pH regulators, surfactants and emulsifiers.

Oral formulations or dosage forms of the DPP-4 inhibitor of this invention may be prepared according to known techniques.

A pharmaceutical composition or dosage form (e.g. oral tablet) of a DPP-4 inhibitor according to embodiment A of the invention may typically contain as excipients (in addition to an active ingredient), for example: one or more diluents, a binder, a disintegrant, and a lubricant, preferably each as disclosed herein-below. In an embodiment, the disintegrant may be optional.

Examples of suitable diluents for compounds according to embodiment A include cellulose powder, calcium hydrogen phosphate, erythritol, low substituted hydroxypropyl cellulose, mannitol, pregelatinized starch or xylitol.

Examples of suitable lubricants for compounds according to embodiment A include talc, polyethyleneglycol, calcium behenate, calcium stearate, hydrogenated castor oil or magnesium stearate.

Examples of suitable binders for compounds according to embodiment A include copovidone (copolymerisates of vinylpyrrolidon with other vinylderivates), hydroxypropyl methylcellulose (HPMC), hydroxypropylcellulose (HPC), polyvinylpyrrolidon (povidone), pregelatinized starch, or low-substituted hydroxypropylcellulose (L-HPC).

Examples of suitable disintegrants for compounds according to embodiment A include corn starch or crospovidone.

Suitable methods of preparing (oral) preparations or dosage forms of the DPP-4 inhibitors according to embodiment A of the invention are:

direct tabletting of the active substance in powder mixtures with suitable tabletting excipients;
granulation with suitable excipients and subsequent mixing with suitable excipients and subsequent tabletting as well as film coating; or
packing of powder mixtures or granules into capsules.

Suitable granulation methods are:

wet granulation in the intensive mixer followed by fluidised bed drying;
one-pot granulation;
fluidised bed granulation; or
dry granulation (e.g. by roller compaction) with suitable excipients and subsequent tabletting or packing into capsules.

An exemplary composition for oral use (e.g. tablet core) of a DPP-4 inhibitor according to embodiment A of the invention comprises the first diluent mannitol, pregelatinized starch as a second diluent with additional binder properties, the binder copovidone, the disintegrant corn starch, and magnesium stearate as lubricant; wherein copovidone and/or corn starch may be optional.

A tablet of a DPP-4 inhibitor according to embodiment A of the invention may be film coated, preferably the film coat comprises hydroxypropylmethylcellulose (HPMC), polyethylene glycol (PEG), talc, titanium dioxide and iron oxide (e.g. red and/or yellow).

For details on dosage forms, formulations and administration of DPP-4 inhibitors of this invention, reference is made to scientific literature and/or published patent documents, particularly to those cited herein.

With respect to the first embodiment (embodiment A), the dosage typically required of the DPP-4 inhibitors mentioned herein in embodiment A when administered intravenously is 0.1 mg to 10 mg, preferably 0.25 mg to 5 mg, and when administered orally is 0.5 mg to 100 mg, preferably 2.5 mg to 50 mg or 0.5 mg to 10 mg, more preferably 2.5 mg to 10 mg or 1 mg to 5 mg, in each case 1 to 4 times a day. Thus, e.g. the dosage of 1-[(4-methyl-quinazolin-2-yl)methyl]-3-methyl-7-(2-butyn-1-yl)-8-(3-(R)-amino-piperidin-1-yl)-xanthine when administered orally is 0.5 mg to 10 mg per patient per day, preferably 2.5 mg to 10 mg or 1 mg to 5 mg per patient per day.

A dosage form prepared with a pharmaceutical composition comprising a DPP-4 inhibitor mentioned herein in embodiment A contain the active ingredient in a dosage range of 0.1-100 mg. Thus, e.g. particular oral dosage strengths of 1-[(4-methyl-quinazolin-2-yl)methyl]-3-methyl-7-(2-butyn-1-yl)-8-(3-(R)-amino-piperidin-1-yl)-xanthine are 0.5 mg, 1 mg, 2.5 mg, 5 mg and 10 mg.

A special embodiment of the DPP-4 inhibitors of this invention refers to those orally administered DPP-4 inhibitors which are therapeutically efficacious at low dose levels, e.g. at oral dose levels <100 mg or <70 mg per patient per day, preferably <50 mg, more preferably <30 mg or <20 mg, even more preferably from 1 mg to 10 mg, particularly from 1 mg to 5 mg (more particularly 5 mg), per patient per day (if required, divided into 1 to 4 single doses, particularly 1 or 2 single doses, which may be of the same size, preferentially, administered orally once- or twice daily (more preferentially once-daily), advantageously, administered at any time of day, with or without food. Thus, for example, the daily oral amount 5 mg BI 1356 can be given in an once daily dosing regimen (i.e. 5 mg BI 1356 once daily) or in a twice daily dosing regimen (i.e. 2.5 mg BI 1356 twice daily), at any time of day, with or without food.

The dosage of the active components in the combinations or compositions in accordance with the present invention may be varied, although the amount of the active ingredients shall be such that a suitable dosage form is obtained. Hence, the selected dosage and the selected dosage form shall depend on the desired therapeutic effect, the route of administration and the duration of the treatment. Dosage ranges for the combination may be from the maximal tolerated dose for the single agent to lower doses.

For further details on angioplasty and/or stents (including drug-eluting stents and drug-coating technology of stents), reference is made to scientific literature and/or published patent documents.

The present invention further provides a certain DPP-4 inhibitor as defined herein (preferably linagliptin, optionally in combination with one or more other active agents) for use in for treating and/or preventing metabolic diseases, particularly diabetes, especially type 2 diabetes mellitus, and/or conditions related thereto (e.g. diabetic complications), in a patient (particularly human patient) having or being at risk of stenosis, blood vessel narrowing, re-narrowing or occlusion, revascularization or restenosis, blood clots, neointimal hyperplasia and/or major adverse cardiac events (MACE), such as e.g. death, myocardial infarction or repeat intervention because of restenosis), e.g. following or associated with (peripheral or coronary) angioplasty (such as e.g. performed with stents, stent implantations, bypass operations or balloon catheters).

The present invention further provides a certain DPP-4 inhibitor as defined herein (preferably linagliptin, optionally in combination with one or more other active agents) for use in for treating and/or preventing metabolic diseases, particularly diabetes, especially type 2 diabetes mellitus, and/or conditions related thereto (e.g. diabetic complications), in a patient (particularly human patient) having or being at risk of restenosis, e.g. following or associated with (peripheral or coronary) angioplasty (such as e.g. performed with stents, stent implantations, bypass operations or balloon catheters).

The present invention further provides a certain DPP-4 inhibitor as defined herein (preferably linagliptin, optionally in combination with one or more other active agents) for use in for treating and/or preventing metabolic diseases, particularly diabetes, especially type 2 diabetes mellitus, and/or conditions related thereto (e.g. diabetic complications), in a patient (particularly human patient) who is indicated for or who has (previously) undergone angioplasty (such as e.g. performed with stents, stent implantations, bypass operations or balloon catheters).

Examples of metabolic disorders or diseases amenable by the therapy of this invention may include, without being limited to, type 1 diabetes, type 2 diabetes, impaired glucose tolerance (IGT), impaired fasting blood glucose (IFG), hyperglycemia, postprandial hyperglycemia, postabsorptive hyperglycemia, latent autoimmune diabetes in adults (LADA), overweight, obesity, dyslipidemia, hyperlipidemia, hypercholesterolemia, hypertriglyceridemia, hyperNEFA-emia, fasting or postprandial hyperlipidemia such as postprandial lipemia (e.g. postprandial hypertriglyceridemia), hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome.

The present invention further relates to a certain DPP-4 inhibitor (preferably linagliptin, optionally in combination with one or more other active agents) for use in at least one of the following methods:
preventing, slowing the progression of, delaying the onset of or treating a metabolic disorder or disease, such as e.g. type 1 diabetes mellitus, type 2 diabetes mellitus, impaired glucose tolerance (IGT), impaired fasting blood glucose (IFG), hyperglycemia, postprandial hyperglycemia, postabsorptive hyperglycemia, latent autoimmune diabetes in adults (LADA), overweight, obesity, dyslipidemia, hyperlipidemia, hypercholesterolemia, hypertriglyceridemia, hyperNEFA-emia, postprandial lipemia (e.g. postprandial hypertriglyceridemia), hypertension, atherosclerosis, endothelial dysfunction, osteoporosis, chronic systemic inflammation, non alcoholic fatty liver disease (NAFLD), retinopathy, neuropathy, nephropathy, polycystic ovarian syndrome, and/or metabolic syndrome;
improving and/or maintaining glycemic control and/or for reducing of fasting plasma glucose, of postprandial plasma glucose, of postabsorptive plasma glucose and/or of glycosylated hemoglobin HbA1c, or preventing, reducing the risk of, slowing the progression of, delaying the onset of or treating worsening or deterioration of glycemic control, need for insulin therapy or elevated HbA1c despite treatment;
preventing, slowing, delaying the onset of or reversing progression from pre-diabetes, impaired glucose tolerance (IGT), impaired fasting blood glucose (IFG), insulin resistance and/or from metabolic syndrome to type 2 diabetes mellitus;
preventing, reducing the risk of, slowing the progression of, delaying the onset of or treating of complications of diabetes mellitus such as micro- and macrovascular diseases, such as nephropathy, micro- or macroalbuminuria, proteinuria, retinopathy, cataracts, neuropathy, learning or memory impairment, neurodegenerative or cognitive disorders, cardio- or cerebrovascular diseases, tissue ischaemia, diabetic foot or ulcus, atherosclerosis, hypertension, endothelial dysfunction, myocardial infarction, acute coronary syndrome, unstable angina pectoris, stable angina pectoris, peripheral arterial occlusive disease, cardiomyopathy, heart failure, heart rhythm disorders, vascular restenosis, and/or stroke;
reducing body weight and/or body fat and/or liver fat and/or intra-myocellular fat or preventing an increase in body weight and/or body fat and/or liver fat and/or intra-myocellular fat or facilitating a reduction in body weight and/or body fat and/or liver fat and/or intra-myocellular fat;
preventing, slowing, delaying the onset of or treating the degeneration of pancreatic beta cells and/or the decline of the functionality of pancreatic beta cells and/or for improving, preserving and/or restoring the functionality of pancreatic beta cells and/or stimulating and/or restoring or protecting the functionality of pancreatic insulin secretion;
preventing, slowing, delaying the onset of or treating non alcoholic fatty liver disease (NAFLD) including hepatic steatosis, non-alcoholic steatohepatitis (NASH) and/or liver fibrosis (such as e.g. preventing, slowing the progression, delaying the onset of, attenuating, treating or reversing hepatic steatosis, (hepatic) inflammation and/or an abnormal accumulation of liver fat);
preventing, slowing the progression of, delaying the onset of or treating type 2 diabetes with failure to conventional antidiabetic mono- or combination therapy;
achieving a reduction in the dose of conventional antidiabetic medication required for adequate therapeutic effect;
reducing the risk for adverse effects associated with conventional antidiabetic medication (e.g. hypoglycemia or weight gain, such as associated with e.g. insulin or sulphonylurea medication); and/or
maintaining and/or improving the insulin sensitivity and/or for treating or preventing hyperinsulinemia and/or insulin resistance;
   in a patient in need thereof (such as e.g. a patient as described herein, for example a patient having diabetes),
   particularly
   in a patient (particularly human patient) who is in need of, who is indicated for or who has (previously) undergone (peripheral or coronary) angioplasty (such as e.g. performed with stents, stent implantations, bypass operations or balloon catheters); and/or
   in a patient (particularly human patient) having or being at risk of restenosis, e.g. following or associated with (peripheral or coronary) angioplasty (such as e.g. performed with stents, stent implantations, bypass operations or balloon catheters); and/or
   in a patient (particularly human patient) having or being at risk of stenosis, blood vessel narrowing, re-narrowing or occlusion, revascularization or restenosis, blood clots, neointimal hyperplasia and/or major adverse cardiac events (MACE), such as e.g. death, myocardial infarction or repeat intervention because of restenosis), e.g. following or associated with (peripheral or coronary) angioplasty (such as e.g. performed with stents, stent implantations, bypass operations or balloon catheters).

As different metabolic or functional disorders often occur simultaneously, it is quite often indicated to combine a number of different active principles with one another. Thus, depending on the functional disorders diagnosed, improved treatment outcomes may be obtained if a DPP-4 inhibitor is combined with one or more active substances customary for the respective disorders, such as e.g. one or more active substances selected from among the other antidiabetic substances, especially active substances that lower the blood sugar level or the lipid level in the blood, raise the HDL level in the blood, lower blood pressure or are indicated in the treatment of atherosclerosis or obesity.

The DPP-4 inhibitors mentioned above—besides their use in mono-therapy—may also be used in conjunction with other active substances, by means of which improved treatment results can be obtained. Such a combined treatment may be given as a free combination of the substances or in the form of a fixed combination, for example in a tablet or capsule. Pharmaceutical formulations of the combination partner needed for this may either be obtained commercially as pharmaceutical compositions or may be formulated by the skilled man using conventional methods. The active substances which may be obtained commercially as pharmaceutical compositions are described in numerous places in the prior art, for example in the list of drugs that appears annually, the “Rote Liste®” of the federal association of the pharmaceutical industry, or in the annually updated compilation of manufacturers' information on prescription drugs known as the “Physicians' Desk Reference”.

Examples of antidiabetic combination partners are metformin; sulphonylureas such as glibenclamide, tolbutamide, glimepiride, glipizide, gliquidon, glibornuride and gliclazide; nateglinide; repaglinide; mitiglinide; thiazolidinediones such as rosiglitazone and pioglitazone; PPAR gamma modulators such as metaglidases; PPAR-gamma agonists such as e.g. rivoglitazone, mitoglitazone, INT-131 and balaglitazone; PPAR-gamma antagonists; PPAR-gamma/alpha modulators such as tesaglitazar, muraglitazar, aleglitazar, indeglitazar and KRP297; PPAR-gamma/alpha/delta modulators such as e.g. lobeglitazone; AMPK-activators such as AICAR; acetyl-CoA carboxylase (ACC1 and ACC2) inhibitors; diacylglycerol-acetyltransferase (DGAT) inhibitors; pancreatic beta cell GCRP agonists such as GPR119 agonists (SMT3-receptor-agonists); 11β-HSD-inhibitors; FGF19 agonists or analogues; alpha-glucosidase blockers such as acarbose, voglibose and miglitol; alpha2-antagonists; insulin and insulin analogues such as human insulin, insulin lispro, insulin glusilin, r-DNA-insulinaspart, NPH insulin, insulin detemir, insulin degludec, insulin tregopil, insulin zinc suspension and insulin glargin; Gastric inhibitory Peptide (GIP); amylin and amylin analogues (e.g. pramlintide or davalintide); GLP-1 and GLP-1 analogues such as Exendin-4, e.g. exenatide, exenatide LAR, liraglutide, taspoglutide, lixisenatide (AVE-0010), LY-2428757 (a PEGylated version of GLP-1), dulaglutide (LY-2189265), semaglutide or albiglutide; SGLT2-inhibitors such as e.g. dapagliflozin, sergliflozin (KGT-1251), atigliflozin, canagliflozin, ipragliflozin, luseogliflozin or tofogliflozin; inhibitors of protein tyrosine-phosphatase (e.g. trodusquemine); inhibitors of glucose-6-phosphatase; fructose-1,6-bisphosphatase modulators; glycogen phosphorylase modulators; glucagon receptor antagonists; phosphoenolpyruvatecarboxykinase (PEPCK) inhibitors; pyruvate dehydrogenasekinase (PDK) inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976) or of serine/threonine kinases; glucokinase/regulatory protein modulators incl. glucokinase activators; glycogen synthase kinase inhibitors; inhibitors of the SH2-domain-containing inositol 5-phosphatase type 2 (SHIP2); IKK inhibitors such as high-dose salicylate; JNK1 inhibitors; protein kinase C-theta inhibitors; beta 3 agonists such as ritobegron, YM 178, solabegron, talibegron, N-5984, GRC-1087, rafabegron, FMP825; aldosereductase inhibitors such as AS 3201, zenarestat, fidarestat, epalrestat, ranirestat, NZ-314, CP-744809, and CT-112; SGLT-1 or SGLT-2 inhibitors; KV 1.3 channel inhibitors; GPR40 modulators such as e.g. [(3S)-6-({2′,6′-dimethyl-4′-[3-(methylsulfonyl)propoxy]biphenyl-3-yl}methoxy)-2,3-dihydro-1-benzofuran-3-yl]acetic acid; SCD-1 inhibitors; CCR-2 antagonists; dopamine receptor agonists (bromocriptine mesylate [Cycloset]); 4-(3-(2,6-dimethylbenzyloxy)phenyl)-4-oxobutanoic acid; sirtuin stimulants; and other DPP IV inhibitors.

Metformin is usually given in doses varying from about 500 mg to 2000 mg up to 2500 mg per day using various dosing regimens from about 100 mg to 500 mg or 200 mg to 850 mg (1-3 times a day), or about 300 mg to 1000 mg once or twice a day, or delayed-release metformin in doses of about 100 mg to 1000 mg or preferably 500 mg to 1000 mg once or twice a day or about 500 mg to 2000 mg once a day. Particular dosage strengths may be 250, 500, 625, 750, 850 and 1000 mg of metformin hydrochloride.

A dosage of pioglitazone is usually of about 1-10 mg, 15 mg, 30 mg, or 45 mg once a day.

Rosiglitazone is usually given in doses from 4 to 8 mg once (or divided twice) a day (typical dosage strengths are 2, 4 and 8 mg).

Glibenclamide (glyburide) is usually given in doses from 2.5-5 to 20 mg once (or divided twice) a day (typical dosage strengths are 1.25, 2.5 and 5 mg), or micronized glibenclamide in doses from 0.75-3 to 12 mg once (or divided twice) a day (typical dosage strengths are 1.5, 3, 4.5 and 6 mg).

Glipizide is usually given in doses from 2.5 to 10-20 mg once (or up to 40 mg divided twice) a day (typical dosage strengths are 5 and 10 mg), or extended-release glibenclamide in doses from 5 to 10 mg (up to 20 mg) once a day (typical dosage strengths are 2.5, 5 and 10 mg).

Glimepiride is usually given in doses from 1-2 to 4 mg (up to 8 mg) once a day (typical dosage strengths are 1, 2 and 4 mg).

A dual combination of glibenclamide/metformin is usually given in doses from 1.25/250 once daily to 10/1000 mg twice daily. (typical dosage strengths are 1.25/250, 2.5/500 and 5/500 mg).

A dual combination of glipizide/metformin is usually given in doses from 2.5/250 to 10/1000 mg twice daily (typical dosage strengths are 2.5/250, 2.5/500 and 5/500 mg).

A dual combination of glimepiride/metformin is usually given in doses from 1/250 to 4/1000 mg twice daily.

A dual combination of rosiglitazone/glimepiride is usually given in doses from 4/1 once or twice daily to 4/2 mg twice daily (typical dosage strengths are 4/1, 4/2, 4/4, 8/2 and 8/4 mg).

A dual combination of pioglitazone/glimepiride is usually given in doses from 30/2 to 30/4 mg once daily (typical dosage strengths are 30/4 and 45/4 mg).

A dual combination of rosiglitazone/metformin is usually given in doses from 1/500 to 4/1000 mg twice daily (typical dosage strengths are 1/500, 2/500, 4/500, 2/1000 and 4/1000 mg).

A dual combination of pioglitazone/metformin is usually given in doses from 15/500 once or twice daily to 15/850 mg thrice daily (typical dosage strengths are 15/500 and 15/850 mg).

The non-sulphonylurea insulin secretagogue nateglinide is usually given in doses from 60 to 120 mg with meals (up to 360 mg/day, typical dosage strengths are 60 and 120 mg); repaglinide is usually given in doses from 0.5 to 4 mg with meals (up to 16 mg/day, typical dosage strengths are 0.5, 1 and 2 mg). A dual combination of repaglinide/metformin is available in dosage strengths of 1/500 and 2/850 mg.

Acarbose is usually given in doses from 25 to 100 mg with meals. Miglitol is usually given in doses from 25 to 100 mg with meals.

Examples of combination partners that lower the lipid level in the blood are HMG-CoA-reductase inhibitors such as simvastatin, atorvastatin, lovastatin, fluvastatin, pravastatin, pitavastatin and rosuvastatin; fibrates such as bezafibrate, fenofibrate, clofibrate, gemfibrozil, etofibrate and etofyllinclofibrate; nicotinic acid and the derivatives thereof such as acipimox; PPAR-alpha agonists; PPAR-delta agonists such as e.g. {4-[(R)-2-ethoxy-3-(4-trifluoromethyl-phenoxy)-propylsulfanyl]-2-methyl-phenoxy}-acetic acid; inhibitors of acyl-coenzyme A:cholesterolacyltransferase (ACAT; EC 2.3.1.26) such as avasimibe; cholesterol resorption inhibitors such as ezetimib; substances that bind to bile acid, such as cholestyramine, colestipol and colesevelam; inhibitors of bile acid transport; HDL modulating active substances such as D4F, reverse D4F, LXR modulating active substances and FXR modulating active substances; CETP inhibitors such as torcetrapib, JTT-705 (dalcetrapib) or compound 12 from WO 2007/005572 (anacetrapib); LDL receptor modulators; MTP inhibitors (e.g. lomitapide); and ApoB100 antisense RNA.

A dosage of atorvastatin is usually from 1 mg to 40 mg or 10 mg to 80 mg once a day.

Examples of combination partners that lower blood pressure are beta-blockers such as atenolol, bisoprolol, celiprolol, metoprolol and carvedilol; diuretics such as hydrochlorothiazide, chlortalidon, xipamide, furosemide, piretanide, torasemide, spironolactone, eplerenone, amiloride and triamterene; calcium channel blockers such as amlodipine, nifedipine, nitrendipine, nisoldipine, nicardipine, felodipine, lacidipine, lercanipidine, manidipine, isradipine, nilvadipine, verapamil, gallopamil and diltiazem; ACE inhibitors such as ramipril, lisinopril, cilazapril, quinapril, captopril, enalapril, benazepril, perindopril, fosinopril and trandolapril; as well as angiotensin II receptor blockers (ARBs) such as telmisartan, candesartan, valsartan, losartan, irbesartan, olmesartan, azilsartan and eprosartan.

A dosage of telmisartan is usually from 20 mg to 320 mg or 40 mg to 160 mg per day.

Examples of combination partners which increase the HDL level in the blood are Cholesteryl Ester Transfer Protein (CETP) inhibitors; inhibitors of endothelial lipase; regulators of ABC1; LXRalpha antagonists; LXRbeta agonists; PPAR-delta agonists; LXRalpha/beta regulators, and substances that increase the expression and/or plasma concentration of apolipoprotein A-I.

Examples of combination partners for the treatment of obesity are sibutramine; tetrahydrolipstatin (orlistat); alizyme (cetilistat); dexfenfluramine; axokine; cannabinoid receptor 1 antagonists such as the CB1 antagonist rimonobant; MCH-1 receptor antagonists; MC4 receptor agonists; NPY5 as well as NPY2 antagonists (e.g. velneperit); beta3-AR agonists such as SB-418790 and AD-9677; 5HT2c receptor agonists such as APD 356 (lorcaserin); myostatin inhibitors; Acrp30 and adiponectin; steroyl CoA desaturase (SCD1) inhibitors; fatty acid synthase (FAS) inhibitors; CCK receptor agonists; Ghrelin receptor modulators; Pyy 3-36; orexin receptor antagonists; and tesofensine; as well as the dual combinations bupropion/naltrexone, bupropion/zonisamide, topiramate/phentermine and pramlintide/metreleptin.

Examples of combination partners for the treatment of atherosclerosis are phospholipase A2 inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976); oxLDL antibodies and oxLDL vaccines; apoA-1 Milano; ASA; and VCAM-1 inhibitors.

Further, the certain DPP-4 inhibitor of this invention may be used in combination with a substrate of DPP-4 (particularly with an anti-inflammatory substrate of DPP-4), which may be other than GLP-1, for the purposes according to the present invention, such substrates of DPP-4 include, for example—without being limited to, one or more of the following:

### Incretins:

Glucagon-like peptide (GLP)-1
Glucose-dependent insulinotropic peptide (GIP)

### Neuroactive:

Substance P
Neuropeptide Y (NPY)
Peptide YY

### Energy Homeostasis:

GLP-2
Prolactin
Pituitary adenylate cyclase activating peptide (PACAP)

### Other Hormones:

PACAP 27
Human chorionic gonadotrophin alpha chain
Growth hormone releasing factor (GHRF)
Luteinizing hormone alpha chain
Insulin-like growth factor (IGF-1)
CCL8/eotaxin
CCL22/macrophage-derived chemokine
CXCL9/interferon-gamma-induced monokine

### Chemokines:

CXCL10/interferon-gamma-induced protein-10
CXCL11/interferon-inducible T cell a chemoattractant
CCL3L1/macrophage inflammatory protein 1alpha isoform
LD78beta
CXCL12/stromal-derived factor 1 alpha and beta

### Other:

Enkephalins, gastrin-releasing peptide, vasostatin-1,
peptide histidine methionine, thyrotropin alpha

Further or in addition, the certain DPP-4 inhibitor of this invention may be used in combination with one or more active substances which are indicated in the treatment of nephropathy, such as selected from diuretics, ACE inhibitors and/or ARBs.

Further or in addition, the certain DPP-4 inhibitor of this invention may be used in combination with one or more active substances which are indicated in the treatment or prevention of cardiovascular diseases or events (e.g. major cardiovascular events).

Moreover, optionally in addition, the certain DPP-4 inhibitor of this invention may be used in combination with one or more antiplatelet agents, such as e.g. (low-dose) aspirin (acetylsalicylic acid), a selective COX-2 or nonselective COX-1/COX-2 inhibitor, or a ADP receptor inhibitor, such as a thienopyridine (e.g. clopidogrel or prasugrel), elinogrel or ticagrelor, or a thrombin receptor antagonist such as vorapaxar.

Yet moreover, optionally in addition, the certain DPP-4 inhibitor of this invention may be used in combination with one or more anticoagulant agents, such as e.g. a heparin, a coumarin (such as warfarin or phenprocoumon), a direct thrombin inhibitor (such as e.g. dabigatran), a pentasaccharide inhibitor of Factor Xa (e.g. fondaparinux) or a direct Faktor Xa inhibitor (such as e.g. rivaroxaban or apixaban or edoxaban or otamixaban).

Still yet moreover, optionally in addition, the certain DPP-4 inhibitor of this invention may be used in combination with one or more agents for the treatment of heart failure.

All patent applications cited herein are hereby incorporated by reference in their entireties.

The present invention is not to be limited in scope by the specific embodiments described herein. Various modifications of the invention in addition to those described herein may become apparent to those skilled in the art from the present disclosure. Such modifications are intended to fall within the scope of the present invention.

Further embodiments, features and advantages of the present invention may become apparent from the following examples. The following examples serve to illustrate, by way of example, the principles of the invention without restricting it.

### EXAMPLES

### Linagliptin Attenuates Vascular Smooth Muscle Cell Proliferation and Neointima Formation after Vascular Injury

### Animals:

6-week-old male C57BL/6 mice were purchased and were housed in a polycarbonate cage with a wooden chip mat on the floor; water was available ad libitum. C57BL/6 mice were divided into 2 groups: control (n=14) and linagliptin-treated (n=14). At age 7 weeks, control mice were fed normal chow (22.6% protein, 53.8% carbohydrate, 5.6% fat, 6.6% mineral and vitamin mixture, and 3.3% fiber; total: 356 kcal/100 g) with vehicle; linagliptin-treated mice were fed normal chow with linagliptin (0.083 g/kg chow, which results in mean plasma levels of 50-150 nM, corresponding to an oral dose of 3 mg/kg/d). The animal room was kept on a 12-h light/dark cycle at a constant temperature (22±1° C.) with relative humidity of 55±5% throughout the experimental period. Endothelial denudation injuries were induced in the femoral artery at age 8 weeks, followed by evaluation of neointimal formation at age 12 weeks.

### Guidewire-induced Endothelial Denudation Injury:

Mouse femoral artery endothelial denudation injury was induced in C57BL/6 mice in the control and linagliptin groups at age 8 weeks, as previously described. Briefly, endovascular injury was induced by 4 passages of a 0.25 mm SilverSpeed-10 hydrophilic guide wire (Micro Therapeutics Inc., Irvine, Calif.) into the left femoral artery. Sham surgery without injury was performed on the contralateral right side. Mice were euthanized 4 weeks after injury and the femoral arteries were isolated for tissue analysis.

### Tissue Preparation and Morphometry:

Following sacrifice, mice were perfused via a cannula in the left ventricle with phosphate-buffered saline for 5 min, followed by 4% paraformaldehyde for 30 min at a pressure of 100 cm H2O. The femoral arteries were embedded in paraffin and cut into 5 μm sections for further analysis. Serial sections of 1.5 mm proximal region from the incision site for the wire insertion were evaluated by staining with Elastica van Gieson stain kit (4033-4037, Muto Pure Chemicals Co., Tokyo, Japan) to visualize the internal elastic lamina.

### Cell Culture:

Rat aortic vascular SMC (VSMC) and mouse aortic VSMC were serum-deprived in Dulbecco's modified Eagle's medium containing 0.1% fetal bovine serum (FBS) for at least 24 h and subjected to mitogenic stimulation with 10% FBS for 24 h with or without 12-h pre-treatment of linagliptin at the indicated concentration BrdU Assay. To evaluate cell proliferation of rat aortic SMC, the bromodeoxyuridine (BrdU) incorporation assay was performed using the Cell Proliferation enzyme-linked immunosorbent assay (ELISA) kit (1647229, Roche Applied Science, Mannheim, Germany) as previously described.

### Results:

The results of the above lead to the following conclusions with reference to the corresponding Figure(s):

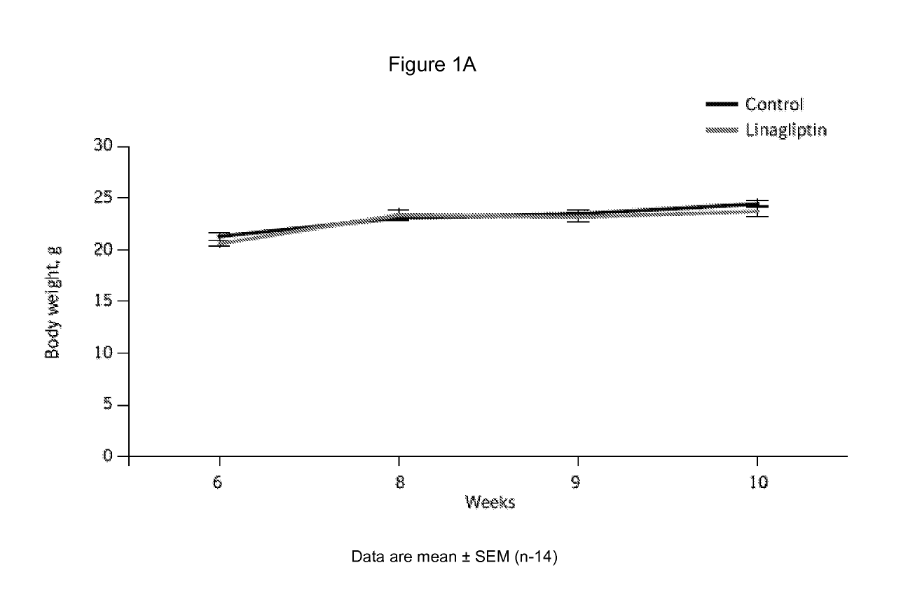

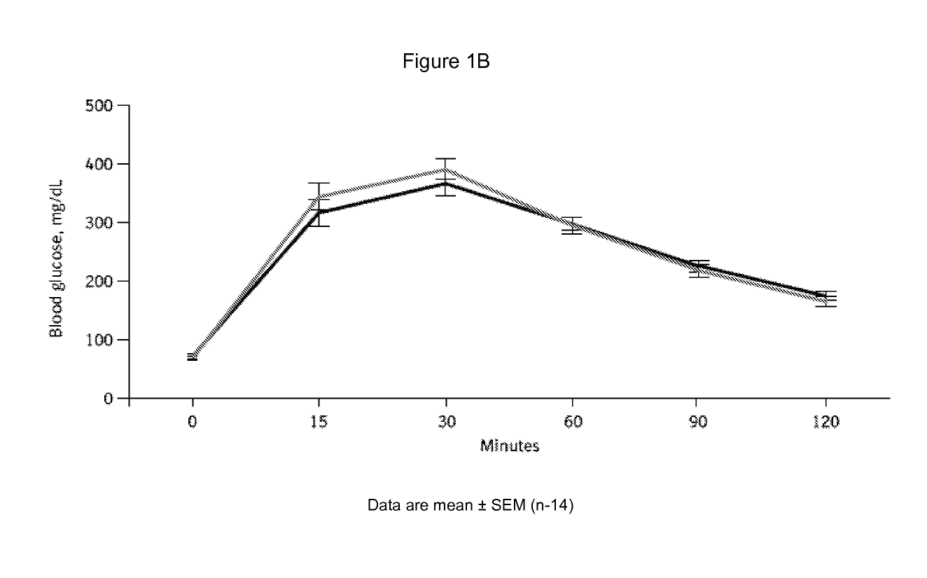

Linagliptin does not change body weight or blood glucose in non-diabetic mice (FIGS. 1A and 1B)

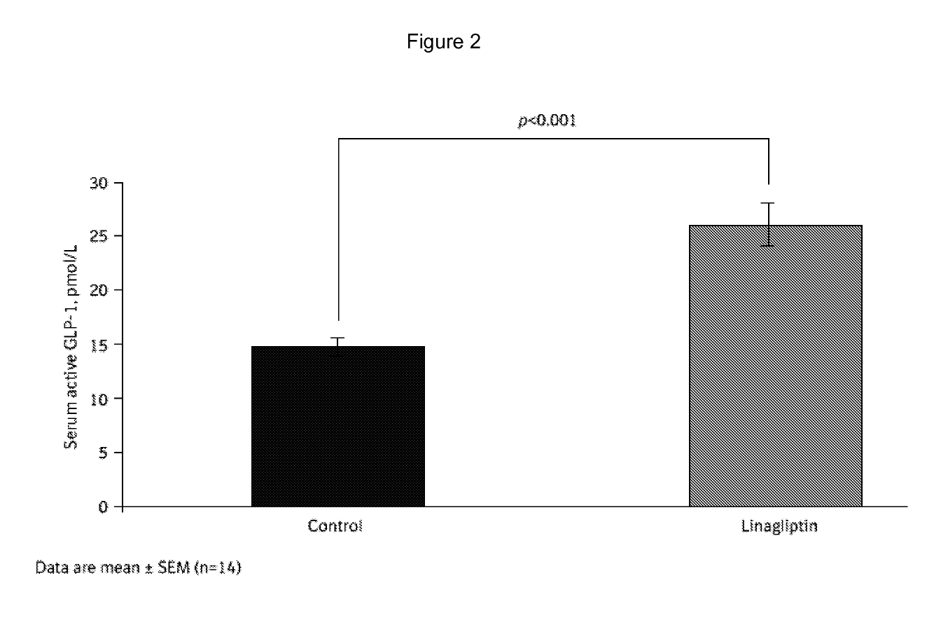

Linagliptin increases serum active GLP-1 concentration (ELISA) in non-diabetic mice (FIG. 2).

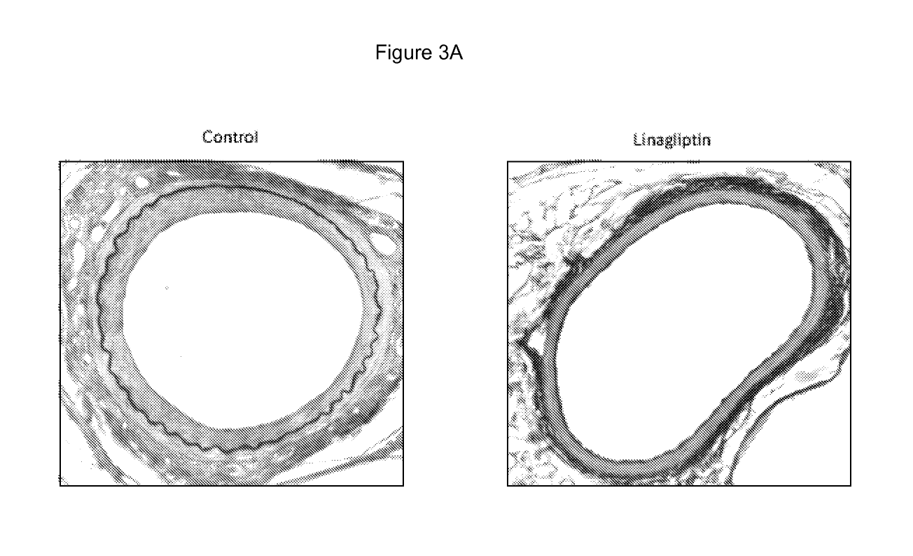

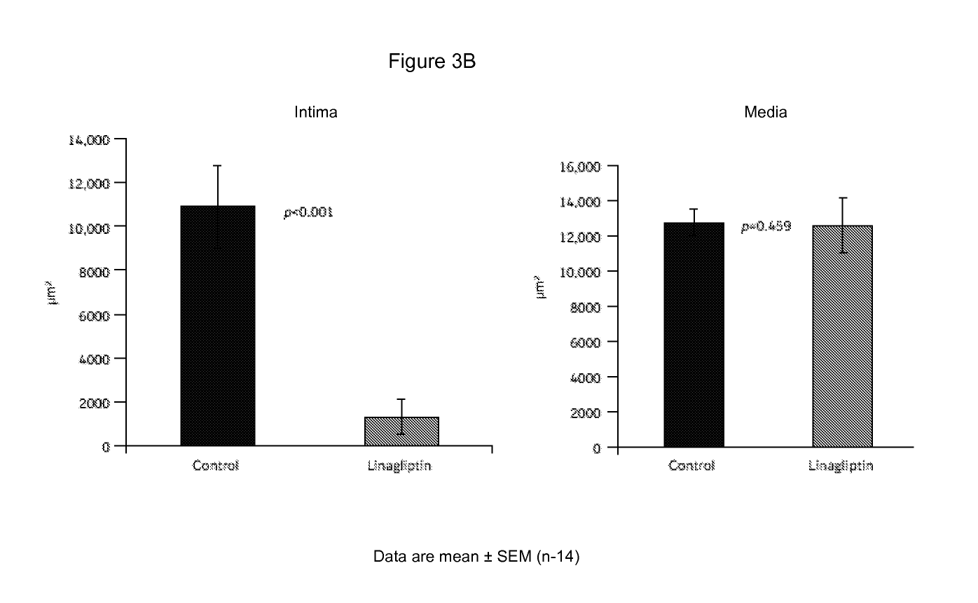

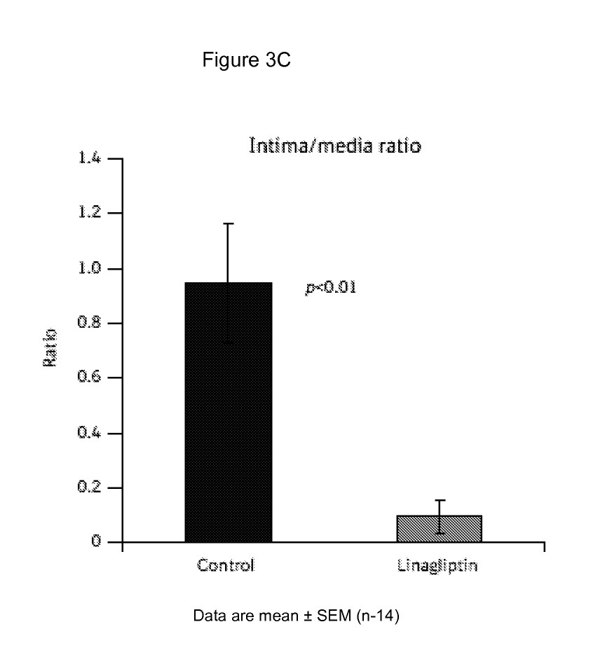

Linagliptin attenuates neointima formation after vascular injury in non-diabetic mice (guide wire and elastic staining) (FIGS. 3A, 3B and 3C).

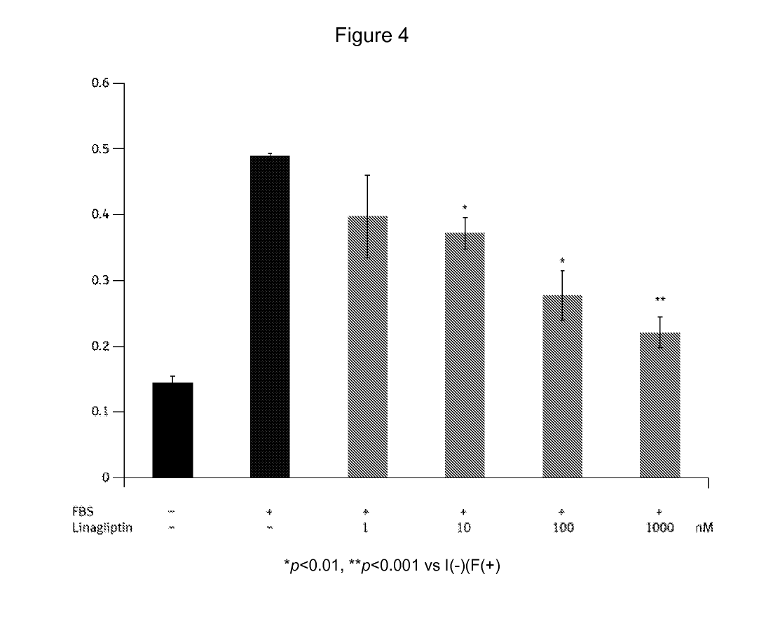

Linagliptin reduces serum-induced vascular smooth muscle (SMC) proliferation in vitro (BrdU assay) (FIG. 4).

1. A method of treating a patient who is in need of, indicated for or has undergone angioplasty and/or stenting, the method comprising administering a DPP-4 inhibitor, optionally in combination with one or more other active agents, to the patient.

2. The method according to claim 1, wherein the DPP-4 inhibitor is used along with angioplasty and/or stenting.

3. The method according to claim 1 for treating, preventing and/or reducing the risk of complications associated with or following angioplasty and/or stenting.

4. The method according to claim 1 for treating, preventing and/or reducing the risk of restenosis from or in angioplasty or stenting.

5. The method according to claim 1, wherein the angioplasty is peripheral or coronary angioplasty.

6. The method according to claim 1, wherein the angioplasty is performed as stent implantation, bypass operation or balloon cathetering.

7. The method according to claim 1, wherein the angioplasty is performed as stent implantation.

8. The method according to claim 1, wherein the DPP-4 inhibitor is used in combination with angioplasty or stenting.

9. The method according to claim 1, wherein the DPP-4 inhibitor is used in combination with a stent.

10. The method according to claim 1, wherein DPP-4 inhibitor is administered orally, optionally in combination with angioplasty or stenting.

11. The method according to claim 1, wherein DPP-4 inhibitor is applied together with a implantable stent.

12. The method according to claim 1, wherein the patient is a diabetic patient.

13. The method according to claim 1, further for treating diabetes.

14. The method according to claim 1, wherein the patient is a non-diabetic patient.

15. The method according to claim 1, wherein the DPP-IV inhibitor is 1-[(4-methyl-quinazolin-2-yl)methyl]-3-methyl-7-(2-butyn-1-yl)-8-(3-(R)-amino-piperidin-1-yl)-xanthine.

16. The method according to claim 3, wherein the one or more complications is selected from the group consisting of selected from restenosis, blood clots, neointimal hyperplasia and major adverse cardiac events (MACE).

17. The method according to claim 16, wherein restenosis is in-stent restenosis (ISR) or post-angioplasty restenosis (PARS).

18. The method according to claim 8, wherein the DPP-4 inhibitor is used simultaneously, concurrently, separately, sequentially or subsequently with angioplasty or stenting.

19. The method according to claim 8, wherein the DPP-4 inhibitor is used in fixed or free combination with a stent.

20. The method according to claim 11, wherein the stent is a drug-eluting stent for release of the DPP-4 inhibitor.
